(19) United States
(12) Patent Application Publication
(10) Pub. No.: US 2026/0160742 A1
(43) Pub. Date: Jun. 11, 2026

(54) WATER QUALITY MEASUREMENT DEVICE AND WATER QUALITY MEASUREMENT SYSTEM

(71) Applicant: MITSUBISHI HEAVY INDUSTRIES, LTD., Tokyo (JP)

(72) Inventors: Nobuyuki UKAI, Tokyo (JP); Naoki OGAWA, Tokyo (JP); Shinichi OKAMOTO, Tokyo (JP); Koudai INOUE, Tokyo (JP); Keiji FUJIKAWA, Tokyo (JP); Takashi IKE, Tokyo (JP)

(73) Assignee: MITSUBISHI HEAVY INDUSTRIES, LTD., Tokyo (JP)

(21) Appl. No.: 19/129,186

(22) PCT Filed: Sep. 7, 2023
(86) PCT No.: PCT/JP2023/032744

§ 371 Date: May 12, 2025

(30) Foreign Application Priority Data
Dec. 1, 2022 (JP) 2022-192578

(51) Int. Cl. G01N 31/16; G01N 33/18

(52) U.S. Cl. CPC G01N 31/16; G01N 33/18

(57) ABSTRACT

A water quality measurement device that measures acid consumption or alkali consumption of a specimen by titration includes: a specimen acquisition device for acquiring the specimen from a supply source of the specimen; a reactor having a reaction chamber communicating with the specimen acquisition device; a dropping part for adding a titrant dropwise to the reaction chamber; a defoamer supply part for supplying a defoamer to the reaction chamber; and a pH measurement device disposed in the reaction chamber for measuring hydrogen ion concentration of the specimen.

### TECHNICAL FIELD

The present disclosure relates to a water quality measurement device and a water quality measurement system that measure the acid consumption or alkali consumption of a specimen by titration.

The present application claims priority based on Japanese Patent Application No. 2022-192578 filed on Dec. 1, 2022, the entire content of which is incorporated herein by reference.

### BACKGROUND ART

Patent Document 1 discloses a water quality measurement method that measures total alkalinity and total carbonic acid concentration in water, especially in seawater. In this water quality measurement method, acid titration is performed with the test water (seawater) being placed in a sealed specimen bottle until a predetermined pH is reached, and total alkalinity and total carbonic acid concentration are determined from the acid titer and pH electrode changes obtained from the results of the acid titration.

### CITATION LIST

### Patent Literature

Patent Document 1: JP2009-264913A

### SUMMARY

### Problems to be Solved

However, when titration (especially acid titration) is performed on a specimen, a large amount of foam may be generated depending on the specimen, which may degrade the measurement accuracy.

The present disclosure was made in view of the above, and an object thereof is to provide a water quality measurement device that measures the acid consumption or alkali consumption of a specimen by titration while improving the measurement accuracy.

### Solution to the Problems

To achieve the above object, a water quality measurement device according to the present disclosure is a water quality measurement device that measures the acid consumption or alkali consumption of a specimen by titration, including: a specimen acquisition device for acquiring the specimen from a supply source of the specimen; a reactor having a reaction chamber communicating with the specimen acquisition device; a dropping part for adding a titrant dropwise to the reaction chamber; a defoamer supply part for supplying a defoamer to the reaction chamber; and a pH measurement device disposed in the reaction chamber for measuring hydrogen ion concentration of the specimen.

### Advantageous Effects

With the water quality measurement device of the present disclosure, it is possible to measure the acid consumption or alkali consumption of a specimen by titration while improving the measurement accuracy.

### BRIEF DESCRIPTION OF DRAWINGS

FIG. 1 is a schematic configuration diagram of a waste treatment facility equipped with a water quality measurement device according to an embodiment.

FIG. 2 is a diagram for describing the configuration of a gas supply pipe of a gas supply part according to some embodiments.

FIG. 3 is a schematic configuration diagram of a water quality measurement system according to another embodiment.

### DETAILED DESCRIPTION

Hereinafter, a water quality measurement device and a water quality measurement system according to embodiments of the present disclosure will be described with reference to the drawings. The following embodiments are illustrative and not intended to limit the present disclosure, and various modifications are possible within the scope of technical ideas of the present disclosure.

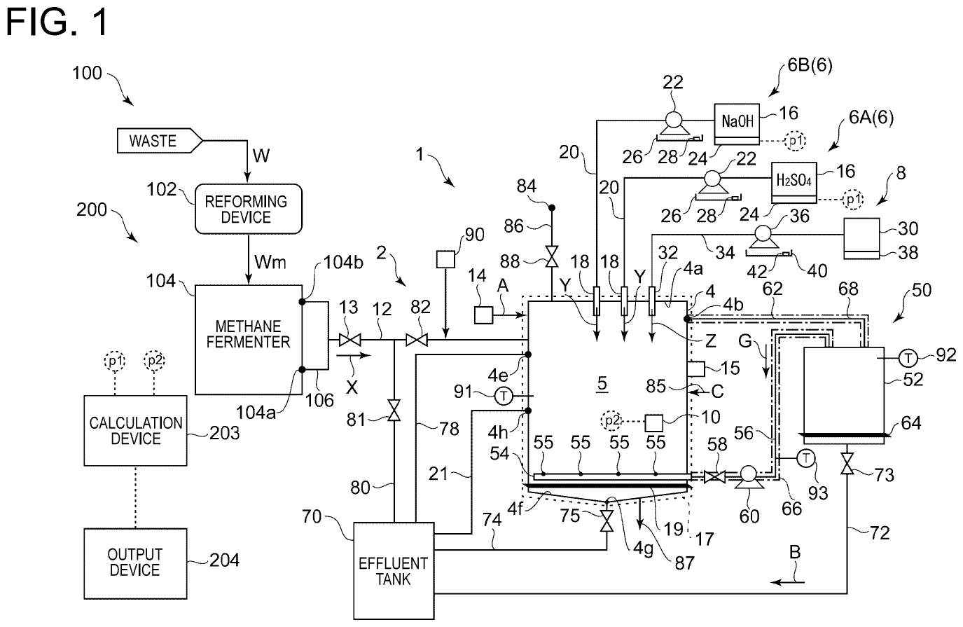

FIG. 1 is a schematic configuration diagram of a waste treatment facility 100 equipped with a water quality measurement device 1 according to an embodiment. In the embodiment illustrated in FIG. 1, the waste treatment facility 100 includes a reforming device 102, a methane fermenter 104, and a circulation line 106.

The reforming device 102 hydrolyzes waste W with steam in a batch manner to produce a reformed material Wm. The waste W is, for example, municipal waste. The municipal waste mainly contains kitchen waste, paper waste, and plastic waste, with a small amount of metal. The present disclosure does not limit the waste W to municipal waste. The waste W may be waste such as sludge generated by treating effluent from factories or the like and agricultural waste with a higher moisture content than municipal waste.

The hydrolysis of the waste W in the reforming device 102 may be wet hydrolysis in which steam contacts the waste W and heats the waste W, or may be dry hydrolysis in which steam indirectly heats the waste W without contacting the waste W.

The methane fermenter 104 is supplied with the reformed material Wm from the reforming device 102 and microbially degrades the supplied reformed material Wm to produce methane gas and methane fermentation liquid X.

The circulation line 106 takes out the contents of the methane fermenter 104 and returns it back to the methane fermenter 104. The circulation line 106 connects a methane fermentation liquid outlet port 104a formed in a lower portion of the methane fermenter 104 to a methane fermentation liquid return port 104b formed in an upper portion of the methane fermenter 104. The circulation line 106 takes the methane fermentation liquid X in the methane fermenter 104 out of the methane fermenter 104 through the methane fermentation liquid outlet port 104a and returns the methane fermentation liquid X taken out of the methane fermenter 104 to the methane fermenter 104 through the methane fermentation liquid return port 104b. In the methane fermenter 104, the methane fermentation liquid X in the methane fermenter 104 is agitated by the circulation line 106.

### Water Quality Measurement Device

### Configuration

The water quality measurement device 1 measures the acid consumption or alkali consumption of a specimen by titration. The specimen may be any solution containing a solvent and solute and is not limited to a particular solution. In an embodiment, as shown in FIG. 1, the water quality measurement device 1 includes a specimen acquisition device 2, a reactor 4, a dropping part 6, a defoamer supply part 8, and a pH measurement device 10.

The specimen acquisition device 2 acquires a specimen (methane fermentation liquid X) from a supply source (circulation line 106) of the specimen. In an embodiment, as illustrated in FIG. 1, the specimen acquisition device 2 includes a supply line 12 connecting the circulation line 106 to the reactor 4, and a supply valve 13 disposed in the supply line 12. When the supply valve 13 is opened, part of the methane fermentation liquid X flowing through the circulation line 106 flows through the supply line 12 to the reactor 4 (a second supply valve 82, which will be described later, is also open). That is, the methane fermentation liquid X is supplied as a specimen to the reaction chamber 5 of the reactor 4. On the other hand, when the supply valve 13 is closed, the supply of the methane fermentation liquid X to the reaction chamber 5 of the reactor 4 is stopped. In an embodiment, the supply line 12 is not equipped with a filtration device (e.g., strainer) that filters the methane fermentation liquid X flowing through the supply line 12. Also, the circulation line 106 is not equipped with a filtration device that filters the methane fermentation liquid X flowing through the circulation line 106, so that the methane fermentation liquid X in the methane fermenter 104 is supplied to the reaction chamber 5 of the reactor 4 without filtration.

The reactor 4 has a cylindrical shape and internally has a reaction chamber 5 communicating with the specimen acquisition device 2. In an embodiment, the reaction chamber 5 communicates with the supply line 12 and is supplied with the methane fermentation liquid X through the supply line 12 as described above.

In an embodiment, as illustrated in FIG. 1, the water quality measurement device 1 further includes a dilution part 14 for supplying dilution water A to the reaction chamber 5. The dilution water A may be, but is not limited to, water which dilutes the methane fermentation liquid X when mixed with the methane fermentation liquid X. Although not shown, in some embodiments, the dilution part 14 may supply the dilution water A to the supply line 12. That is, the dilution part 14 may supply the dilution water A to the reaction chamber 5 via the supply line 12.

In an embodiment, as illustrated in FIG. 1, the water quality measurement device 1 further includes a level meter sensor 15 for detecting the position of the liquid surface of the methane fermentation liquid X in the reaction chamber 5. This facilitates adjustment of the amount of methane fermentation liquid X and dilution water A supplied to the reaction chamber 5 and allows easy preparation of a specimen of the methane fermentation liquid X diluted at any ratio or adjusted to any pH. In some embodiments, the water quality measurement device 1 measures the acid consumption or alkali consumption of the methane fermentation liquid X diluted two-fold.

In an embodiment, as illustrated in FIG. 1, the water quality measurement device 1 further includes a metering adjustment line 21 connected to the reactor 4. The metering adjustment line 21 is configured to be able to switch whether or not to discharge the methane fermentation liquid X from the reaction chamber 5 when the liquid surface of the methane fermentation liquid X in the reaction chamber 5 exceeds a predetermined height. In the embodiment illustrated in FIG. 1, the metering adjustment line 21 connects the reactor 4 to an effluent tank 70, which will be described below. The metering adjustment line 21 communicates with the reaction chamber 5 through an opening 4h that can be opened and closed in the side wall of the reactor 4. The opening 4h is located below an overflow port 4e, which will be described below.

With this configuration, by opening the opening 4h, the amount of methane fermentation liquid X supplied to the reaction chamber 5 can be adjusted to a fixed amount corresponding to the height at which the opening 4h is located, so that the fixed amount of methane fermentation liquid X can be easily prepared. After the fixed amount of methane fermentation liquid X is prepared, the dilution water A may be supplied to the reaction chamber 5 to easily prepare a specimen of the methane fermentation liquid X diluted at any ratio.

In an embodiment, as illustrated in FIG. 1, the water quality measurement device 1 further includes a reaction chamber heat insulator 17 for keeping the reaction chamber 5 warm and a reaction chamber heating device 19 for heating the reaction chamber 5. The reaction chamber heat insulator 17 (shown by the dotted line in FIG. 1) surrounds the perimeter of the reactor 4 and reduces heat transfer between the reaction chamber 5 (the inside of the reactor 4) and the outside of the reactor 4. The material of the reaction chamber heat insulator 17 is flexible and insulating, for example, glass wool. The reaction chamber heating device 19 is, for example, a wound heater that is wound around the reactor 4. Although not shown, in some embodiments, the water quality measurement device 1 includes either the reaction chamber heat insulator 17 or the reaction chamber heating device 19.

The dropping part 6 is configured to add a titrant Y of predetermined concentration dropwise to the reaction chamber 5. In an embodiment, as illustrated in FIG. 1, the dropping part 6 includes a first dropping part 6A (6) that adds sulfuric acid (H₂SO₄) as the titrant Y dropwise to the reaction chamber 5 and a second dropping part 6B (6) that adds sodium hydroxide solution (NaOH) as the titrant Y dropwise to the reaction chamber 5. That is, the first dropping part 6A is used for acid titration to measure the acid consumption (alkalinity) of the methane fermentation liquid X. Similarly, the second dropping part 6B is used for alkaline titration to measure the alkali consumption (acidity) of the methane fermentation liquid X.

The first dropping part 6A includes a titrant reservoir 16 for storing sulfuric acid (titrant Y), a dropping nozzle 18 fitted into an upper wall 4a of the reactor 4, a titrant line 20 connecting the titrant reservoir 16 to the dropping nozzle 18, and a titrant pump 22 disposed in the titrant line 20. By driving the titrant pump 22, sulfuric acid flows through the titrant line 20 from the titrant reservoir 16 to the dropping nozzle 18. Sulfuric acid is then added dropwise into the reaction chamber 5 through the dropping nozzle 18.

In the embodiment illustrated in FIG. 1, the first dropping part 6A further includes a first load cell 24 for measuring the weight of the titrant reservoir 16. By acquiring a measured value of the first load cell 24, the amount of sulfuric acid (titrant Y) added to the reaction chamber 5 can be easily calculated.

In the embodiment illustrated in FIG. 1, the first dropping part 6A further includes a first drip pan 26 disposed below the titrant pump 22 to receive sulfuric acid (titrant Y) that leaks from the titrant pump 22, and a first leak sensor 28 for detecting sulfuric acid in the first drip pan 26. The titrant pump 22 may develop leaks, for example, due to aging of the packing. Therefore, by providing the first drip pan 26 and the first leak sensor 28, leakage of sulfuric acid from the titrant pump 22 can be quickly detected.

The second dropping part 6B has the same configuration as the first dropping part 6A and is marked with the same reference numerals as those of the first dropping part 6A, and detailed description will be omitted. In the second dropping part 6B, “sulfuric acid” in the description of the first dropping part 6A is replaced with “sodium hydroxide solution”. In an embodiment, the water quality measurement device 1 includes two dropping parts 6 (first dropping part 6A, second dropping part 6B) to measure the acid consumption and alkali consumption of the methane fermentation liquid X. However, the present disclosure is not limited to this embodiment. In some embodiments, the water quality measurement device 1 may include one dropping part 6 where acid titration or alkali titration is performed.

The defoamer supply part 8 is configured to supply a defoamer Z to the reaction chamber 5. The defoamer Z serves to break the foam generated when the titrant Y is added dropwise to the methane fermentation liquid X (especially in acid titration) or foam generated by aeration as described below. The defoamer Z is, for example, an oil-based defoamer or a surfactant-based defoamer. Such defoamer Z may be added in advance to the methane fermentation liquid X before the dropwise addition of the titrant Y or before the aeration to suppress the generation of foam.

In an embodiment, as illustrated in FIG. 1, the defoamer supply part 8 includes a defoamer reservoir 30 for storing the defoamer Z, a defoamer nozzle 32 fitted into the upper wall 4a of the reactor 4, a defoamer line 34 connecting the defoamer reservoir 30 to the defoamer nozzle 32, and a defoamer pump 36 disposed in the defoamer line 34. By driving the defoamer pump 36, the defoamer Z flows through the defoamer line 34 from the defoamer reservoir 30 to the defoamer nozzle 32. The defoamer Z is then supplied to the reaction chamber 5 through the defoamer nozzle 32. That is, the defoamer nozzle 32 is disposed in an upper portion of the reactor 4 to spray the defoamer Z from above the methane fermentation liquid X (specimen) in the reaction chamber 5.

In the embodiment illustrated in FIG. 1, the defoamer supply part 8 further includes a second load cell 38 for measuring the weight of the defoamer reservoir 30. By acquiring a measured value of the second load cell 38, the amount of defoamer Z supplied to the reaction chamber 5 can be easily calculated.

In the embodiment illustrated in FIG. 1, the defoamer supply part 8 further includes a second drip pan 40 disposed below the defoamer pump 36 to receive the defoamer Z that leaks from the defoamer pump 36, and a second leak sensor 42 for detecting the defoamer Z in the second drip pan 40. The defoamer pump 36 may develop leaks, for example, due to aging of the packing. Therefore, by providing the second drip pan 40 and the second leak sensor 42, leakage of the defoamer Z from the defoamer pump 36 can be quickly detected.

The pH measurement device 10 is disposed within the reaction chamber 5 to measure the hydrogen ion concentration of the methane fermentation liquid X (specimen). The pH measurement device 10 is located below the overflow port 4e, which will be described below.

In an embodiment, as illustrated in FIG. 1, the water quality measurement device 1 further includes a gas supply part 50 for causing a gas G to flow through the methane fermentation liquid X (specimen) in the reaction chamber 5. The gas G is not limited as long as it is in gaseous form and may be, for example, air, carbon dioxide gas, or nitrogen gas.

In the embodiment illustrated in FIG. 1, the gas supply part 50 includes a buffer tank 52 for storing the gas G, a gas supply pipe 54 which passes through a lower portion of the reaction chamber 5 and inside which the G gas flows, and a gas supply line 56 connecting the buffer tank 52 to the gas supply pipe 54. The gas supply pipe 54 has a gas supply hole 55 allowing the gas G to flow out to the reaction chamber 5.

The gas supply line 56 is equipped with a gas valve 58 and a gas pump 60. When the gas valve 58 is opened and the gas pump 60 is driven, the gas G flows through the gas supply line 56 from the buffer tank 52 to the gas supply pipe 54. The gas G then flows out to the reaction chamber 5 through the gas supply hole 55. The gas G flowing out of the gas supply hole 55 agitates the methane fermentation liquid X in the reaction chamber 5. That is, the gas supply part 50 aerates the methane fermentation liquid X in the reaction chamber 5.

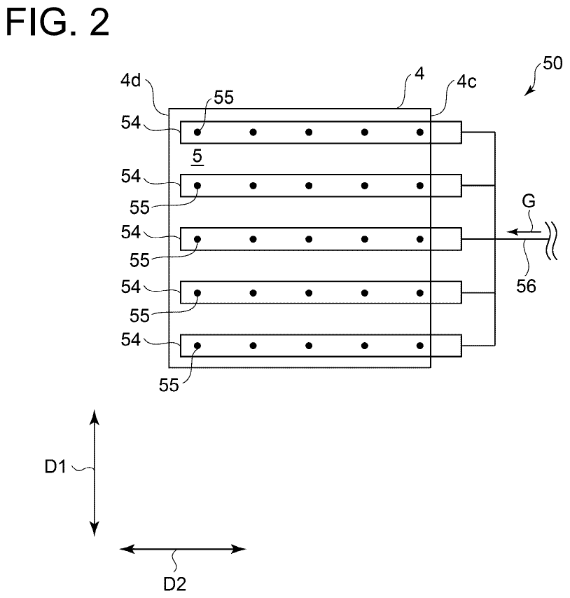

FIG. 2 is a diagram for describing the configuration of the gas supply pipe 54 of the gas supply part 50 according to some embodiments when the reaction chamber 5 is viewed from above. As illustrated in FIG. 2, in some embodiments, the gas supply part 50 includes a plurality of gas supply pipes 54 arranged at intervals along one direction D1 within the horizontal direction. In the crossing direction D2, which intersects with one direction D1, when a second side wall 4d is opposite a first side wall 4c of the reactor 4 into which the gas supply pipes 54 are fitted, each of the plurality of gas supply pipes 54 has a tip located near the second side wall 4d. Each of the plurality of gas supply pipes 54 has a plurality of gas supply holes 55 arranged at intervals along the crossing direction D2. With this configuration, the gas supply part 50 can perform full aeration, where the methane fermentation liquid X in the reaction chamber 5 is agitated as a whole.

In the embodiment illustrated in FIG. 1, the gas supply part 50 further includes a gas outlet line 62, a heating device 64, and a heat insulator 66.

The gas outlet line 62 is connected at one end to a gas outlet port 4b formed in an upper portion of the reactor 4 and at the other end to the buffer tank 52. The gas outlet port 4b is located above the overflow port 4e, which will be described below. When the air pressure in the reaction chamber 5 is higher than the air pressure in the gas outlet line 62, the gas G in the reaction chamber 5 flows through the gas outlet line 62 toward the buffer tank 52. Thus, the gas supply line 56 and the gas outlet line 62 constitute a gas circulation line 68 for sucking the gas G in the reaction chamber 5 and returning the gas G to the reaction chamber 5. Further, the buffer tank 52 stores a liquid phase B generated by the condensation of the gas G flowing through the gas outlet line 62 or the gas G in the buffer tank 52. That is, the buffer tank 52 functions as a vessel to separate the liquid phase B from the gas G sucked from inside the reaction chamber 5. In some embodiments, the buffer tank 52 includes a pressure reducing part for reducing the internal pressure and a demister disposed within the tank.

The heating device 64 heats the gas G flowing through the gas circulation line 68. In an embodiment, as illustrated in FIG. 1, the heating device 64 is disposed on the buffer tank 52 to heat the gas G in the buffer tank 52. The heating device 64 is, for example, a wound heater that is wound around the buffer tank 52 and heats the gas G stored in the buffer tank 52. In some embodiments, the heating device 64 is disposed in the gas supply line 56 or the gas outlet line 62.

The heat insulator 66 keeps the gas G flowing through the gas circulation line 68 warm. In an embodiment, as illustrated in FIG. 1, the heat insulator 66 (shown by the dotted and dashed line in FIG. 1) surrounds the perimeter of the gas supply line 56 and the gas outlet line 62 and reduces heat transfer between the inside and outside of the gas supply line 56 and heat transfer between the inside and outside of the gas outlet line 62. The material of the heat insulator 66 is flexible and insulating, for example, glass wool. In some embodiments, the heat insulator 66 surrounds the perimeter of the gas supply line 56.

In an embodiment, as illustrated in FIG. 1, the water quality measurement device 1 further includes an effluent tank 70 for storing the methane fermentation liquid X (specimen) discharged from the reactor 4, and a liquid phase discharge line 72 connecting the effluent tank 70 to the buffer tank 52. In the embodiment illustrated in FIG. 1, the water quality measurement device 1 includes a first line 74 which connects a bottom portion of the reactor 4 to the effluent tank 70 and through which the methane fermentation liquid X flows. The first line 74 is equipped with a first valve 75. When the first valve 75 is opened, the methane fermentation liquid X flows through the first line 74 from the reactor 4 to the effluent tank 70. The liquid phase discharge line 72 connects a bottom portion of the buffer tank 52 to the effluent tank 70. The liquid phase discharge line 72 is equipped with a liquid phase discharge valve 73. When the liquid phase discharge valve 73 is opened, the liquid phase B flows through the liquid phase discharge line 72 from the buffer tank 52 to the effluent tank 70.

In the embodiment illustrated in FIG. 1, the water quality measurement device 1 includes a second line 78 which connects the overflow port 4e formed on the side wall of the reactor 4 to the effluent tank 70. By forming the overflow port 4e, the methane fermentation liquid X can be discharged from the reactor 4 when the liquid surface of the methane fermentation liquid X in the reaction chamber 5 exceeds a predetermined height.

In the embodiment illustrated in FIG. 1, the water quality measurement device 1 further includes a third line 80 connected at one end to the supply line 12 at a position downstream (reactor 4 side) of the supply valve 13 and at the other end to the effluent tank 70, and a third valve 81 disposed in the third line 80. In this case, the supply line 12 is further equipped with a second supply valve 82 downstream of the connection point of the third line 80. Before measuring the methane fermentation liquid X with the water quality measurement device 1, the supply valve 13 and the third valve 81 are opened to transfer the methane fermentation liquid X to the effluent tank 70. After a certain amount of the methane fermentation liquid X is transferred to the effluent tank 70, the second supply valve 82 is opened and the third valve 81 is closed, so that all of the methane fermentation liquid X flowing into the supply line 12 is supplied to the reactor 4. This reduces the time required to supply the methane fermentation liquid X to the reactor 4.

In the embodiment illustrated in FIG. 1, the water quality measurement device 1 further includes an air line 86 open at one end to the atmosphere and connected at the other end to the reactor 4, and an air valve 88 disposed in the air line 86. After the water quality measurement device 1 completes the water quality measurement of the methane fermentation liquid X, the air valve 88 is opened to release the reaction chamber 5 from the sealed state and allow the methane fermentation liquid X in the reaction chamber 5 to be discharged into the effluent tank 70.

In the embodiment illustrated in FIG. 1, the reactor 4 includes a discharge port 4g formed on a lower wall 4f. The bottom surface of the lower wall 4f facing the reaction chamber 5 slopes downward toward the discharge port 4g. The first line 74 communicates with the reaction chamber 5 through the discharge port 4g. With this configuration, the methane fermentation liquid X can be smoothly discharged from the reaction chamber 5. In the embodiment illustrated in FIG. 1, the entire bottom surface of the lower wall 4f slopes, but the present disclosure is not limited to this embodiment. In some embodiments, a portion of the bottom surface of the lower wall 4f slopes.

In the embodiment illustrated in FIG. 1, the water quality measurement device 1 further includes a calibration fluid supply part 85 for supplying a calibration fluid C of known concentration to the reaction chamber 5, and a calibration fluid discharge part 87 for discharging the calibration fluid C from the reaction chamber 5. The calibration fluid C is, for example, sulfuric acid or sodium hydroxide aqueous solution whose concentration has already been measured. With this configuration, it is easy to check at any given time whether the measurement accuracy of the water quality measurement device 1 is properly maintained.

In the embodiment illustrated in FIG. 1, the water quality measurement device 1 includes a defoamer addition device 90 for adding the defoamer Z to the methane fermentation liquid X flowing through the supply line 12. The defoamer addition device 90 supplies the defoamer Z to the supply line 12 at a position downstream of the second supply valve 82.

In the embodiment illustrated in FIG. 1, the water quality measurement device 1 further includes a first thermometer 91 disposed on the reactor 4 to acquire the temperature in the reactor 4, a second thermometer 92 disposed on the buffer tank 52 to acquire the temperature in the buffer tank 52, and a third thermometer 93 disposed on the gas supply line 56 to acquire the temperature in the gas supply line 56.

In the embodiment illustrated in FIG. 1, the waste treatment facility 100 includes a water quality measurement system 200 having the above-described water quality measurement device 1, methane fermenter 104, and circulation line 106. The water quality measurement system 200 further includes a calculation device 203 and an output device 204. The calculation device 203 is electrically connected to the first load cell 24 (p1) and can acquire a measured value of the first load cell 24. The calculation device 203 calculates the amount of titrant Y added to the reaction chamber 5 on the basis of a change in the measured value of the first load cell 24. The calculation device 203 is electrically connected to the pH measurement device 10 (p2) and can acquire a measured value of the pH measurement device 10. The calculation device 203 calculates the acid consumption or alkali consumption of the methane fermentation liquid X on the basis of the amount of titrant Y added to the reaction chamber 5 and the measured value of the pH measurement device. The output device 204 is, for example, a display, which is electrically connected to the calculation device 203 and acquires a calculated value of the calculation device 203. The output device 204 outputs the acid consumption or alkali consumption of the methane fermentation liquid X calculated by the calculation device 203.

The calculation device 203 may acquire the temperature in the reactor 4 from the first thermometer 91 and calculate the acid consumption or alkali consumption of the methane fermentation liquid X by taking this temperature into account. The calculation device 203 may acquire a measured value of the second load cell 38 and calculate the acid consumption or alkali consumption of the methane fermentation liquid X by taking into account the amount of defoamer Z calculated from the measured value of the second load cell 38.

### Operation and Effect

The operation and effect of the water quality measurement device 1 according to an embodiment will be described. According to an embodiment, the methane fermentation liquid X obtained from the circulation line 106 without filtration is supplied to the reaction chamber 5. The acid consumption and alkali consumption can be measured by adding the titrant Y (sulfuric acid and sodium hydroxide solution, respectively) dropwise to the methane fermentation liquid X. Further, by supplying the defoamer Z to the methane fermentation liquid X in the reaction chamber 5, the foam generated during the dropwise addition of the titrant Y or aeration can be broken, and the effects of foam, such as leakage of the methane fermentation liquid X due to the generation of a large amount of foam, can be reduced. Therefore, it is possible to measure the acid consumption and alkali consumption of the unfiltered methane fermentation liquid X by titration while improving the measurement accuracy of the water quality measurement device 1.

According to an embodiment, since the methane fermentation liquid X is aerated by the gas supply part 50, the reaction between the methane fermentation liquid X and the titrant Y is accelerated when the titrant Y is added dropwise to the methane fermentation liquid X, and the measurement accuracy of the water quality measurement device 1 can be improved. Further, when the defoamer Z is supplied to the methane fermentation liquid X, the mixing of the methane fermentation liquid X and the defoamer Z is accelerated, and the generation of foam can be reduced. It is also possible to agitate highly viscous methane fermentation liquid X. The present disclosure does not limit the method of agitating the methane fermentation liquid X to aeration. Although not shown, in some embodiments, the water quality measurement device 1 may be equipped with a rotating body, such as a magnetic stirrer, in the reaction chamber 5 for agitating the methane fermentation liquid X.

According to an embodiment, since aeration is implemented by allowing the gas G to flow out of the gas supply hole 55 into the reaction chamber 5, the gas G can flow out from any position in the reaction chamber 5 to achieve any aeration, such as full aeration. This further accelerates the reaction between the methane fermentation liquid X and the titrant Y and the mixing of the methane fermentation liquid X and the defoamer Z.

According to an embodiment, the buffer tank 52 is provided to ensure the amount of the gas G to perform aeration. Further, the buffer tank 52 is provided with the heating device 64 to ensure the amount of the gas G heated. Further, the liquid phase B separated from the gas G can be stored in the buffer tank 52.

The humidity of the gas G sucked from inside the reaction chamber 5 is high, and the moisture in the gas G may condense before returning the gas G to the reaction chamber 5. According to an embodiment, the buffer tank 52 is provided to separate the liquid phase B from the gas G sucked from inside the reaction chamber 5, reducing the occurrence of problems in the downstream of the buffer tank 52, such as blockage of the gas supply line 56, blockage of the gas valve 58, and failure of the gas pump 60 due to the liquid phase B.

According to an embodiment, the gas circulation line 68 allows the gas G to flow through a closed system. Therefore, unlike an open system, the gas G is prevented from flowing to the outside, and adverse effects such as bad odors are reduced.

The measured value of the water quality measurement device 1 (the amount of titrant Y dropped) may be affected by the temperature of the methane fermentation liquid X. If the temperature of the reaction chamber 5 drops, the measurement accuracy of the water quality measurement device 1 may degrade. According to an embodiment, the gas G heated in the buffer tank 52 is supplied to the reaction chamber 5. Additionally, the gas supply line 56 and the gas outlet line 62 are provided with the heat insulator 66, which reduces changes in the temperature of the gas G flowing through each of the gas supply line 56 and the gas outlet line 62. As a result, the temperature of the reaction chamber 5 can be maintained, and a decrease in the measurement accuracy of the water quality measurement device 1 can be suppressed.

According to an embodiment, the liquid phase B stored in the buffer tank 52 can be discharged into the effluent tank 70 through the liquid phase discharge line 72. This allows the combined disposal of the methane fermentation liquid X and the liquid phase B. Although not shown, in some embodiments, the water quality measurement device 1 further includes a level meter sensor for detecting the position of the liquid level of the liquid (methane fermentation liquid X+liquid phase B) in the effluent tank 70, and the effluent tank 70 is configured such that the liquid in the effluent tank 70 is discharged when the detected liquid level exceeds a predetermined height.

According to an embodiment, by providing the reaction chamber heat insulator 17 and the reaction chamber heating device 19, the temperature of the reaction chamber 5 can be maintained at a temperature suitable for titration, and the measurement accuracy of the water quality measurement device 1 can be improved. Additionally, since the gas G circulates between the reactor 4 and the buffer tank 52, the change in the output of each of the reaction chamber heating device 19 and the heating device 64 can be minimized. Specifically, there is no need to adjust the output, such as decreasing the output in the summer and increasing the output in the winter. Additionally, by checking the first thermometer 91, the second thermometer 92, and the third thermometer 93, it is possible to check whether the gas G is maintained at a constant temperature, and if a temperature difference is observed, the output of the reaction chamber heating device 19 or the heating device 64 can be adjusted to maintain the gas G at a constant temperature.

According to an embodiment, the methane fermentation liquid X is diluted with dilution water A to reduce the viscosity of the methane fermentation liquid X and facilitate agitation of the methane fermentation liquid X. This further accelerates the reaction between the methane fermentation liquid X and the titrant Y and the mixing of the methane fermentation liquid X and the defoamer Z. The dilution of the methane fermentation liquid X with dilution water A also facilitates breaking the foam by the defoamer Z.

According to an embodiment, since the defoamer nozzle 32 is fitted into the upper wall 4a of the reactor 4, the defoamer Z can be directly sprinkled on the foam generated on the surface of the methane fermentation liquid X in the reaction chamber 5 to break the foam and effectively reduce the effects of foam.

According to an embodiment, by using an oil-based defoamer or a surfactant-based defoamer, the defoamer Z is highly effective in breaking the foam generated by the dropwise addition of the titrant Y to the methane fermentation liquid X or aeration of the methane fermentation liquid X.

According to an embodiment, the methane fermentation liquid X in the methane fermenter 104 is agitated by the circulation line 106. Then, the methane fermentation liquid X is obtained from the circulation line 106. This makes it possible to evaluate the state of the methane fermentation liquid X, for example, by preventing the acquisition of an excessive amount of supernatant of the methane fermentation liquid X in the methane fermenter 104 or sludge contained in the methane fermentation liquid X.

According to an embodiment, the defoamer addition device 90 is provided so that the defoamer Z is added in advance to the methane fermentation liquid X before the dropwise addition of the titrant Y or before the aeration. This reduces the generation of foam by the dropwise addition of the titrant Y to the methane fermentation liquid X or aeration of the methane fermentation liquid X.

In an embodiment, the water quality measurement device 1 measures the acid consumption and alkali consumption of the methane fermentation liquid X in the methane fermenter 104, but the present disclosure is not limited to this embodiment. In some embodiments, the water quality measurement device 1 measures the acid consumption or alkali consumption of the contents of a fermenter that produces biogas other than methane as a valuable resource from the reformed material Wm by biological action of microorganisms.

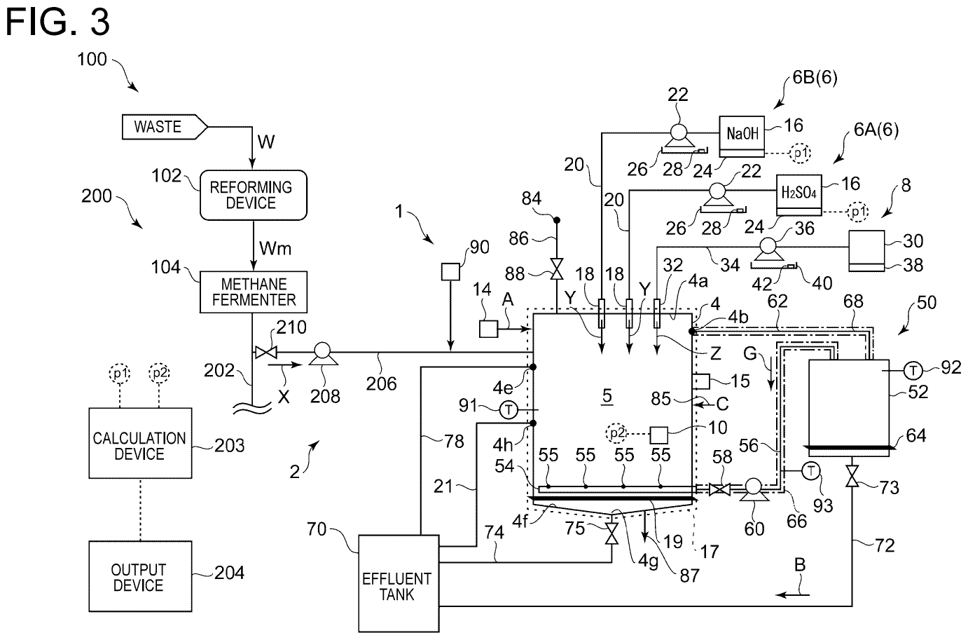

In an embodiment, the specimen acquisition device 2 acquires the methane fermentation liquid X from the circulation line 106, but the present invention is not limited to this embodiment. FIG. 3 is a schematic configuration diagram of a water quality measurement system 200 according to another embodiment.

As illustrated in FIG. 3, in another embodiment, the water quality measurement system 200 includes a water quality measurement device 1, a methane fermenter 104, and an extraction line 202. In the embodiment illustrated in FIG. 3, the water quality measurement device 1 has the same configuration as the water quality measurement device 1 illustrated in FIG. 1 except for the specimen acquisition device 2, and the same reference numerals as those shown in FIG. 1 are used, and detailed description will be omitted. In the embodiment illustrated in FIG. 3, the methane fermenter 104 has the same configuration as the methane fermenter 104 illustrated in FIG. 1.

The extraction line 202 is connected at one end to a bottom portion of the methane fermenter 104 and at the other end to a device (not shown) other than the methane fermenter 104. The extraction line 202 can extract the methane fermentation liquid X in the methane fermenter 104. For example, the methane fermentation liquid X in the methane fermenter 104 is extracted by opening a valve (not shown) in the extraction line 202. The methane fermentation liquid X may be extracted from the methane fermenter 104 periodically or at any time. For example, the methane fermentation liquid X is extracted from the methane fermenter 104 once per day.

In another embodiment, as illustrated in FIG. 3, the specimen acquisition device 2 includes a supply line 206 and a pump 208.

The supply line 206 connects the extraction line 202 to the reactor 4. The supply line 206 has a hole diameter of 12.7 mm or more. The pump 208 is disposed in the supply line 206. In the embodiment illustrated in FIG. 3, the specimen acquisition device 2 has a supply valve 210 in the supply line 206, and when the supply valve 210 is opened and the pump 208 is driven, the methane fermentation liquid X flows through the supply line 206 from the extraction line 202 to the reactor 4.

With the configuration illustrated in FIG. 3, the acid consumption and alkali consumption of the methane fermentation liquid X can be measured when the methane fermentation liquid X is extracted from the methane fermenter 104. Further, by setting the hole diameter of the supply line 206 to 12.7 mm or more, the supply line 206 is prevented from being clogged with sludge contained in the methane fermentation liquid X.

The contents described in the above embodiments would be understood as follows, for instance.

[1] A water quality measurement device (1) according to the present disclosure is a water quality measurement device that measures acid consumption or alkali consumption of a specimen (methane fermentation liquid X) by titration, including: a specimen acquisition device (2) for acquiring the specimen from a supply source (circulation line 106, extraction line 202) of the specimen; a reactor (4) having a reaction chamber (5) communicating with the specimen acquisition device; a dropping part (6) for adding a titrant (Y) dropwise to the reaction chamber; a defoamer supply part (8) for supplying a defoamer (Z) to the reaction chamber; and a pH measurement device (10) disposed in the reaction chamber for measuring hydrogen ion concentration of the specimen.

With the above configuration [1], the acid consumption or alkali consumption can be measured by supplying the specimen acquired from the supply source of the specimen to the reaction chamber, and adding the titrant dropwise to the specimen in the reaction chamber. Further, by supplying the defoamer to the specimen in the reaction chamber, the effects of foam generated during the dropwise addition of the titrant to the specimen can be reduced. Thus, it is possible to measure the acid consumption or alkali consumption of the specimen by titration while improving the measurement accuracy.

[2] In some embodiments, in the above configuration [1], the water quality measurement device further includes a gas supply part (50) for causing a gas (G) to flow through the specimen in the reaction chamber.

With the above configuration [2], the specimen is agitated, so that the reaction between the specimen and the titrant is accelerated when the titrant is added dropwise to the specimen. Further, when the defoamer is supplied to the specimen, the mixing of the specimen and the defoamer is accelerated.

[3] In some embodiments, in the above configuration [2], the gas supply part includes at least one gas supply pipe (54) which passes through the reaction chamber and inside which the gas flows. The at least one gas supply pipe has at least one gas supply hole (55) allowing the gas to flow out to the reaction chamber.

With the above configuration [3], since the gas can flow out from any position in the reaction chamber, the reaction between the specimen and the titrant and the mixing of the specimen and the defoamer are further accelerated.

[4] In some embodiments, in the above configuration [2] or [3], the gas supply part includes a gas circulation line (68) for sucking the gas in the reaction chamber and returning the gas to the reaction chamber.

With the above configuration [4], the gas circulation line allows the gas to flow through a closed system. Therefore, unlike an open system, the gas is prevented from flowing to the outside, and adverse effects such as bad odors are reduced.

[5] In some embodiments, in the above configuration [4], the gas supply part further includes a heating device (64) for heating the gas flowing through the gas circulation line.

The measured value of the water quality measurement device may be affected by the temperature of the specimen. If the temperature of the reaction chamber drops, the measurement accuracy may degrade. With the above configuration [5], since the gas heated by the heating device flows through the reaction chamber, the temperature of the reaction chamber can be maintained, and a decrease in the measurement accuracy can be suppressed.

[6] In some embodiments, in the above configuration [5], the gas supply part further includes a gas-liquid separation device (buffer tank 52) for separating a liquid phase from the gas flowing through the gas circulation line.

The humidity of the gas sucked from inside the reaction chamber is high. With the above configuration [5], since the liquid phase is separated from the gas sucked from inside the reaction chamber, the effects of the liquid phase can be reduced.

[7] In some embodiments, in the above configuration [6], the gas-liquid separation device includes a buffer tank (52) for storing the gas. The heating device is disposed on the buffer tank to heat the gas in the buffer tank.

With the above configuration [7], the amount of heated gas flowing in the reaction chamber can be ensured. Further, the liquid phase separated from the gas can be stored in the buffer tank.

[8] In some embodiments, in the above configuration [7], the gas supply part further includes a gas pump (60) disposed in the gas circulation line to cause the gas in the buffer tank to flow from the buffer tank into the reactor.

With the above configuration [8], the gas heated by the heating device can flow into the reactor in any amount and for any period of time.

[9] In some embodiments, in the above configuration [7] or [8], the water quality measurement device further includes an effluent tank (70) for storing the specimen discharged from the reactor; and a liquid phase discharge line (72) connecting the effluent tank to the buffer tank.

With the above configuration [7], the liquid phase stored in the buffer tank can be discharged into the effluent tank through the liquid phase discharge line. This allows the combined disposal of the specimen and the liquid phase.

[10] In some embodiments, in any one of the above configurations [4] to [9], the gas supply part further includes a heat insulator (66) for keeping the gas flowing through the gas circulation line warm.

With the above configuration [10], the heat insulator reduces changes in the temperature of the gas flowing through the gas circulation line. As a result, the temperature of the reaction chamber can be maintained, and a decrease in the measurement accuracy can be suppressed.

[11] In some embodiments, in any one of the above configurations [1] to [10], the water quality measurement device further includes at least one of a reaction chamber heat insulator (17) for keeping the reaction chamber warm or a reaction chamber heating device (19) for heating the reaction chamber.

With the above configuration [11], the temperature of the reaction chamber can be maintained at a temperature suitable for titration, and the measurement accuracy can be improved.

[12] In some embodiments, in any one of the above configurations [1] to [11], the water quality measurement device further includes a dilution part (14) for supplying dilution water (A) to the reaction chamber.

With the above configuration [12], it is possible to reduce the viscosity of the specimen and facilitate agitation of the specimen. This further accelerates the reaction between the specimen and the titrant and the mixing of the specimen and the defoamer.

[13] In some embodiments, in any one of the above configurations [1] to [12], the defoamer supply part includes a defoamer nozzle (32) disposed in an upper portion of the reactor to spray the defoamer from above the specimen in the reaction chamber.

With the above configuration [13], the defoamer can be directly sprinkled on foam generated on the top surface of the specimen in the reaction chamber to break the foam and effectively reduce the effects of foam.

[14] In some embodiments, in any one of the above configurations [1] to [13], the defoamer is an oil-based defoamer or a surfactant-based defoamer.

With the above configuration [14], it is possible to achieve a defoamer that is highly effective in breaking the foam.

[15] In some embodiments, in any one of the above configurations [1] to [14], the water quality measurement device further includes a thermometer (91) disposed on the reactor for acquiring temperature in the reactor.

With the above configuration [15], it is possible to check whether the temperature in the reaction chamber is maintained at a constant temperature. If the temperature in the reaction chamber is not maintained at a constant temperature, the process is immediately performed to maintain the temperature in the reaction chamber at a constant temperature to achieve good temperature conditions.

[16] In some embodiments, in any one of the above configurations [1] to [15], the water quality measurement device further includes a level meter sensor (15) for detecting a position of a liquid surface of the specimen in the reaction chamber.

With the above configuration [16], it becomes easier to adjust the amount of the specimen in the reaction chamber, and it is possible to easily prepare any amount of the specimen.

[17] In some embodiments, in the above configuration [16], the water quality measurement device further includes a metering adjustment line (21) connected to the reactor. The metering adjustment line is configured to be able to switch whether or not to discharge the specimen from the reaction chamber when the liquid surface of the specimen in the reaction chamber exceeds a predetermined height.

With the above configuration [17], the metering adjustment line can adjust the amount of the specimen in the reaction chamber to a fixed amount where the liquid level of the specimen is at a predetermined height. This makes it easy to prepare a fixed amount of the specimen.

[18] In some embodiments, in any one of the above configurations [1] to [17], the reactor has an overflow port (4e) formed on a side wall of the reactor and located above the pH measurement device.

With the above configuration [18], the specimen in the reaction chamber can be discharged from the reactor through the overflow port before the specimen overflows from the reactor.

[19] In some embodiments, in any one of the above configurations [1] to [18], the water quality measurement device further includes: a calibration fluid supply part (85) for supplying a calibration fluid of known concentration to the reaction chamber; and a calibration fluid discharge part (87) for discharging the calibration fluid from the reaction chamber.

With the above configuration [19], since the calibration fluid can be supplied and discharged to/from the reaction chamber, it is easy to check at any given time whether the measurement accuracy of the water quality measurement device is properly maintained.

[20] In some embodiments, in any one of the above configurations [1] to [19], the dropping part includes: a titrant reservoir (16) which stores the titrant; a dropping nozzle (18) fitted into the reactor; a titrant line (20) connecting the titrant reservoir to the dropping nozzle; and a titrant pump (22) disposed in the titrant line.

With the above configuration [20], the titrant can be added dropwise to the reaction chamber.

[21] In some embodiments, in the above configuration [20], the dropping part further includes a weight measurement device (24) for measuring weight of the titrant reservoir.

With the above configuration [21], by acquiring a measured value of the weight measurement device, the amount of titrant Y added to the reaction chamber can be easily calculated.

[22] In some embodiments, in the above configuration [20] or [21], the dropping part further includes: a drip pan (26) disposed below the titrant pump to receive the titrant that leaks from the titrant pump; and a leak sensor (28) for detecting the titrant in the drip pan.

With the above configuration [22], leakage of the titrant from the titrant pump can be quickly detected.

[23] In some embodiments, in any one of the above configurations [1] to [22], the reactor includes a discharge port (4g) formed on a lower wall (4f) of the reactor, and a bottom surface of the lower wall facing the reaction chamber slopes downward toward the discharge port.

With the above configuration [23], the specimen can be smoothly discharged from the reaction chamber.

[24] A water quality measurement system according to some embodiments includes:
the water quality measurement device in any one of the above configurations [1] to [23]; a fermenter (methane fermenter 104) for microbially degrading a reformed material (Wm) obtained by hydrolysis of waste (W), the fermenter being the supply source; and a circulation line (106) for taking out contents (methane fermentation liquid X) of the fermenter and returning the contents to the fermenter. The specimen acquisition device includes a supply line (12) connecting the circulation line to the reactor and allowing the contents of the fermenter flowing through the circulation line to flow toward the reactor.

With the above configuration [24], since the contents of the fermenter are acquired as the specimen from the circulation line, the bias of the contents can be reduced, and the condition of the contents of the fermenter can be evaluated.

[25] A water quality measurement system according to some embodiments includes:
the water quality measurement device in any one of the above configurations [1] to [23]; a fermenter for microbially degrading a reformed material obtained by hydrolysis of waste, the fermenter being the supply source; and an extraction line (202) connected to a bottom portion of the fermenter and capable of extracting contents of the fermenter. The specimen acquisition device includes: a supply line (206) connecting the extraction line to the reactor; and a pump (208) disposed in the supply line to cause the contents of the fermenter to flow from the extraction line into the reactor.

With the above configuration [25], the acid consumption or alkali consumption of the contents of the fermenter can be measured when the contents are extracted from the fermenter.

[26] In some embodiments, in the above configuration [25], the supply line has a hole diameter of 12.7 mm or more.

With the above configuration [26], by setting the hole diameter of the supply line to 12.7 mm or more, the supply line is prevented from being clogged with the contents of the fermenter.

[27] In some embodiments, in any one of the above configurations [24] to [26], the water quality measurement device includes a defoamer addition device (90) for adding the defoamer to the specimen flowing through the supply line.

With the above configuration [27], by adding the defoamer to the specimen before the dropwise addition of the titrant to the specimen, the generation of foam during titration can be suppressed.

[28] In some embodiments, in any one of the above configurations [24] to [27], the water quality measurement system further includes a calculation device (203) for calculating the acid consumption or alkali consumption of the specimen on the basis of amount of the titrant added dropwise to the reaction chamber and a measured value of the pH measurement device.

With the above configuration [28], the acid consumption or alkali consumption of the specimen can be automatically calculated.

[29] In some embodiments, in the above configuration [28], the water quality measurement system further includes an output device (204) for outputting the acid consumption or alkali consumption of the specimen calculated by the calculation device.

With the above configuration [29], the acid consumption or alkali consumption of the specimen can be quickly determined.

### REFERENCE SIGNS LIST

1 Water quality measurement device
2 Specimen acquisition device
4 Reactor
4a Wall
4b Gas outlet port
4c First side wall
4d Second side wall
4e Overflow port
4f Lower wall
4g Discharge port
5 Reaction chamber
6 Dropping part
6A First dropping part
6B Second dropping part
8 Defoamer supply part
10 pH measurement device
12 Supply line
13 Supply valve
14 Dilution part
15 Level meter sensor
16 Titrant reservoir
17 Reaction chamber heat insulator
18 Dropping nozzle
19 Reaction chamber heating device
20 Titrant line
21 Metering adjustment line
22 Titrant pump
24 First load cell
26 First drip pan
28 First leak sensor
30 Defoamer reservoir
32 Defoamer nozzle
34 Defoamer line
36 Defoamer pump
38 Second load cell
40 Second drip pan
42 Second leak sensor
50 Gas supply part
52 Buffer tank
54 Gas supply pipe
55 Gas supply hole
56 Gas supply line
58 Gas valve
60 Gas pump
62 Gas outlet line
64 Heating device
66 Heat insulator
68 Gas circulation line
70 Effluent tank
72 Liquid phase discharge line
73 Liquid phase discharge valve
74 First line
75 First valve
78 Second line
80 Third line
81 Third valve
82 Second supply valve
85 Calibration fluid supply part
86 Air line
87 Calibration fluid discharge part
88 Air valve
90 Defoamer addition device
91 First thermometer
92 Second thermometer
93 Third thermometer
100 Waste treatment facility
102 Reforming device
104 Methane fermenter
104a Methane fermentation liquid outlet port
104b Methane fermentation liquid return port
106 Circulation line
200 Water quality measurement system
202 Extraction line
203 Calculation device
204 Output device
206 Supply line
208 Pump
210 Supply valve
A Dilution water
B Liquid phase
D1 One direction
D2 Crossing direction
G Gas
W Waste
Wm Reformed material
X Methane fermentation liquid
Y Titrant
Z Defoamer

1. A water quality measurement device that measures acid consumption or alkali consumption of a specimen by titration, comprising:
a specimen acquisition device for acquiring the specimen from a supply source of the specimen;
a reactor having a reaction chamber communicating with the specimen acquisition device;
a dropping part for adding a titrant dropwise to the reaction chamber;
a defoamer supply part for supplying a defoamer to the reaction chamber; and
a pH measurement device disposed in the reaction chamber for measuring hydrogen ion concentration of the specimen.

2. The water quality measurement device according to claim 1, further comprising a gas supply part for causing a gas to flow through the specimen in the reaction chamber.

3. The water quality measurement device according to claim 2,
wherein the gas supply part includes at least one gas supply pipe which passes through the reaction chamber and inside which the gas flows, and
wherein the at least one gas supply pipe has at least one gas supply hole allowing the gas to flow out to the reaction chamber.

4. The water quality measurement device according to claim 2,
wherein the gas supply part includes a gas circulation line for sucking the gas in the reaction chamber and returning the gas to the reaction chamber.

5-10. (canceled)

11. The water quality measurement device according to claim 1, further comprising at least one of a reaction chamber heat insulator for keeping the reaction chamber warm or a reaction chamber heating device for heating the reaction chamber.

12. The water quality measurement device according to claim 1, further comprising a dilution part for supplying dilution water to the reaction chamber.

13. The water quality measurement device according to claim 1,
wherein the defoamer supply part includes a defoamer nozzle disposed in an upper portion of the reactor to spray the defoamer from above the specimen in the reaction chamber.

14. (canceled)

15. The water quality measurement device according to claim 1, further comprising a thermometer disposed on the reactor for acquiring temperature in the reactor.

16. The water quality measurement device according to claim 1, further comprising a level meter sensor for detecting a position of a liquid surface of the specimen in the reaction chamber.

17. The water quality measurement device according to claim 16, further comprising a metering adjustment line connected to the reactor,
wherein the metering adjustment line is configured to be able to switch whether or not to discharge the specimen from the reaction chamber when the liquid surface of the specimen in the reaction chamber exceeds a predetermined height.

18. The water quality measurement device according to claim 1,
wherein the reactor has an overflow port formed on a side wall of the reactor and located above the pH measurement device.

19. The water quality measurement device according to claim 1, further comprising:
a calibration fluid supply part for supplying a calibration fluid of known concentration to the reaction chamber; and
a calibration fluid discharge part for discharging the calibration fluid from the reaction chamber.

20. The water quality measurement device according to claim 1,
wherein the dropping part includes:
a titrant reservoir which stores the titrant;
a dropping nozzle fitted into the reactor;
a titrant line connecting the titrant reservoir to the dropping nozzle; and
a titrant pump disposed in the titrant line.

21. The water quality measurement device according to claim 20,
wherein the dropping part further includes a weight measurement device for measuring weight of the titrant reservoir.

22. (canceled)

23. The water quality measurement device according to claim 1,
wherein the reactor includes a discharge port formed on a lower wall of the reactor, and
wherein a bottom surface of the lower wall facing the reaction chamber slopes downward toward the discharge port.

24. A water quality measurement system, comprising:
the water quality measurement device according to claim 1,
a fermenter for microbially degrading a reformed material obtained by hydrolysis of waste, the fermenter being the supply source; and
a circulation line for taking out contents of the fermenter and returning the contents to the fermenter,
wherein the specimen acquisition device includes a supply line connecting the circulation line to the reactor and allowing the contents of the fermenter flowing through the circulation line to flow toward the reactor.

25. A water quality measurement system, comprising:
the water quality measurement device according to claim 1,
a fermenter for microbially degrading a reformed material obtained by hydrolysis of waste, the fermenter being the supply source; and
an extraction line connected to a bottom portion of the fermenter and capable of extracting contents of the fermenter,
wherein the specimen acquisition device includes:
a supply line connecting the extraction line to the reactor; and
a pump disposed in the supply line to cause the contents of the fermenter to flow from the extraction line into the reactor.

26. The water quality measurement system according to claim 25,
wherein the supply line has a hole diameter of 12.7 mm or more.

27. The water quality measurement system according to claim 24,
wherein the water quality measurement device includes a defoamer addition device for adding the defoamer to the specimen flowing through the supply line.

28. The water quality measurement system according to claim 24, further comprising a calculation device for calculating the acid consumption or alkali consumption of the specimen on the basis of amount of the titrant added dropwise to the reaction chamber and a measured value of the pH measurement device.

29. (canceled)
